(19) United States
(12) Patent Application Publication
(10) Pub. No.: US 2008/0208149 A1
(43) Pub. Date: Aug. 28, 2008

(54) Male apron for receiving and storing excreted body fluids and associated method

(76) Inventor: Joe Vasquez, San Diego, CA (US)

Correspondence Address: ASHKAN NAJAFI, P.A., 6817 SOUTHPOINT PARKWAY, SUITE 2301, JACKSONVILLE, FL, 32216, US

(21) Appl. No.: 12/070,773

(22) Filed: Feb. 21, 2008

Related U.S. Application Data
(60) Provisional application No. 60/902,910, filed on Feb. 23, 2007.

(51) Int. Cl. A61M 1/00; A41B 13/10

(52) U.S. Cl. 604/322; 2/48

(57) ABSTRACT

A urine storing apron includes a flexible belt, a flexible apron coupled to the belt, a plurality of pouches attached to anterior and posterior sides of the apron, and a mechanism for storing the body fluid inside one of the pouches. The apparatus further includes a mechanism for simultaneously discharging air and the body fluid from the one pouch by rotationally articulating a portion of the simultaneous discharge mechanism about an axis registered orthogonal to an exiting travel path of the body fluid.

### CROSS REFERENCE TO RELATED APPLICATIONS

This application claims the benefit of U.S. Provisional Application No. 60/902,910, filed Feb. 23, 2007, the entire disclosures of which are incorporated herein by reference.

### STATEMENT REGARDING FEDERALLY SPONSORED RESEARCH OR DEVELOPMENT

Not Applicable.

### REFERENCE TO A MICROFICHE APPENDIX

Not Applicable.

### BACKGROUND OF THE INVENTION

1. Technical Field

This invention relates to excretion storage and, more particularly, to a male apron for receiving and storing excreted body fluids while assisting a user to relieve himself from body fluids on-demand.

2. Prior Art

There are no acceptable bladder relief systems for incontinent adults. Urinary incontinence affects more than 13 million Americans in community and institutional settings. Thirty-eight percent of non-institutionalized patients older than 60 years of age experienced urinary incontinence, and almost 50 percent of institutionalized patients. The annual costs of bladder control problems in the United States for people older than 65 years of age was estimated at $26.3 billion in 1995, or $3,565 per affected person. Many incontinent males use commercially available diapers, which cannot contain urine from multiple urinations, and become heavy and uncomfortable when wet. Based on the above mentioned needs, it would be advantageous to provide males who suffer from bladder control problems with a comfortable, private means of relieving themselves when a restroom is not readily available.

U.S. Pat. No. 5,947,942 to Galjour discloses a colostomy bag belt which utilizes: an inner comfort belt which holds a skin patch against the wearer's skin, the skin patch being connected to a connector forming a conduit through the comfort belt; an exterior belt which forms a pouch therein and has a leak-proof safety pouch within the belt and has a connector forming a conduit through the exterior belt and into the safety pouch and which has a sealable opening through which as colostomy bag can be inserted into the safety pouch and secured to the connector; a flexible intermediate connector which completes the conduit from the skin patch to the safety pouch; and a colostomy bag placed in the safety pouch and connected to the conduit to receive the waste which exits the stoma and passes through the conduit. Unfortunately, this prior art example is not designed to accommodate the specific needs of a male.

U.S. Pat. No. 5,653,701 to Millman discloses a ostomy support belt which includes a main pocket for supporting the weight of a conventional pouch for holding bodily drainages. A secondary pocket is provided on the inside of the ostomy support belt in which a support plate is inserted for providing additional lateral support to the areas of the skin and abdomen surrounding the stoma and to increase adhesion where the pouch attaches to the skin surrounding the stoma and to prevent leakage, skin irritation, mounding, and herniation. A guard may be attached to the outside of the belt at a location corresponding to the stoma to prevent the stoma from damage due to external forces. Unfortunately, this prior art example is not designed specifically to accommodate the needs of a male user.

U.S. Pat. No. 5,234,420 to Horton discloses an apparatus for supporting a drainage catheter collection chamber to a body portion of a patient in a comfortable, non-irritating manner including a sheet of soft fiber material having a loop portion formed on at least one edge thereof, at least two tabs extending from corner portions of the loop portion, male snap fastener portions affixed to the loop portion, and female snap fastener portions affixed to the tabs in such a manner that when the tabs are folded over the loop portion the male and female snap fastener portions are in alignment to be engaged with each other. Unfortunately, this prior art example is not designed specifically to accommodate the needs of a male user.

Accordingly, the present invention is disclosed in order to overcome the above noted shortcomings. The urine storing apron is convenient and easy to use, lightweight yet durable in design, and designed for assisting a user to relieve himself from body fluids on-demand. The apparatus is simple to use, inexpensive, and designed for many years of repeated use.

### BRIEF SUMMARY OF THE INVENTION

In view of the foregoing background, it is therefore an object of the present invention to provide an apparatus for assisting a user to relieve himself from body fluids on-demand. These and other objects, features, and advantages of the invention are provided by a urine storing apron.

A urine storing apron includes a flexible belt, a flexible apron coupled to the belt and extending downwardly therefrom, and a plurality of pouches effectively attached to anterior and posterior sides of the apron.

The apparatus further includes a mechanism for storing the body fluid inside one of the pouches. Such a body fluid storing mechanism includes a flexible sack seated within the one pouch and conveniently adaptable for receiving and storing the body fluid therein. Such a flexible sack has an open top end contiguously oriented with a top opening of the one pouch for receiving the body fluid therethrough, and further has an open bottom end disposed subjacent to a bottom-most end of the one pouch for directing the body fluid away from the one pouch. An upper portion of the sack houses the body fluid and is hidden inside of the one pouch such that the body fluid is not visible from an exterior of the one pouch while the open bottom end of the sack is visible from exterior of the one pouch.

The apparatus further includes a mechanism for simultaneously discharging air and the body fluid from the one pouch by rotationally articulating a portion of the simultaneous discharge mechanism about an axis registered orthogonal to an exiting travel path of the body fluid. Such a simultaneous discharging mechanism advantageously includes a bidirectional valve with a hollow chamber intermediately configured between the open top and bottom ends of the sack. Such a chamber has a circular passageway formed therein, and the chamber further has oppositely seated inlet and outlet ports wherein the inlet port is in fluid communication with the upper portion of the sack and the outlet portion is in fluid communication with the open bottom end of the sack respectively.

The simultaneous discharging mechanism further includes a drive shaft medially housed at a center of the bidirectional valve and effectively disposed exterior of the passageway in such a manner that the drive shaft is equidistantly offset from an inner perimeter of the chamber. A drum is concentrically attached directly to the drive shaft and journaled thereabout while remaining spaced inwardly from the inner perimeter of the chamber, and a plurality of non-linear arms has opposed proximal and distal ends directly engaged with the drum and partially seated within the passageway respectively. A plurality of paddles is anchored to distal ends of the arms and contiguously brushes along a major portion of an outer perimeter of the passageway. Also, a crank handle is statically mated with the drum and configured in such a manner that an outer edge of the crank handle extends beyond said outer perimeter of said chamber to thereby uniformly articulate the drum along clockwise and counter clockwise directions based upon a user input. The outlet port of the chamber is importantly located upstream of the open bottom end of the sack for effectively expelling the body fluid downwardly towards a converging path prior to exiting the sack and thereby ensuring that the body fluid is effectively discharged. Such an arrangement between the outlet port and the open bottom end of the sack is critical for ensuring the body fluid is discharged along a concentrated path and not a diverging path that will make it difficult to discharge the body fluid in space confined areas such as a toilet opening or a urinal, for example.

The paddles and the arms rotate in sync and thereby expel the air and the body fluid along the clockwise and counter clockwise directions so that the user can effectively discharge a residual quantity of the body fluid from the sack and reduce the likelihood of undesirable odor and contamination over extended periods of time. Further, each of the paddles are statically coupled to corresponding ones of the arms and are conveniently provided with curvilinear front and rear sides for effectively forcing the air and the body fluid from the inlet port to the outlet port while traveling along the passageway. The inlet and outlet ports are diametrically offset from a center of the chamber such that the air and the body fluid travel approximately half-way along the passageway after entering the inlet port and exiting through the outlet port. Each of the paddles and the arms are coextensively shaped for evenly discharging the air and the body fluid along the chamber while remaining spaced from the inlet and outlet ports respectively.

A method for assisting a user to collect and safely discharge body fluids on-demand includes the steps of: providing a flexible belt; providing a flexible apron coupled to the belt and extending downwardly therefrom; providing a plurality of pouches attached to anterior and posterior sides of the apron; storing the body fluid inside one of the pouches; and simultaneously discharging air and the body fluid from the one pouch by providing and rotationally articulating a bidirectional valve about an axis registered orthogonal to an exiting travel path of the body fluid.

The method further includes the steps of: providing a flexible sack seated within the one pouch and adaptable for receiving and storing the body fluid therein; and depositing the body fluid through an open top end contiguously oriented with a top opening of the one pouch.

The method further includes the steps of: providing a hollow chamber intermediately configured between the open top and bottom ends of the sack and having a circular passageway formed therein; and providing inlet and outlet ports in fluid communication with the upper portion of the sack and the bottom end of the sack respectively.

The method further includes the steps of: providing a drive shaft medially housed at a center of the bidirectional valve by disposing the drive shaft exterior of the passageway in such a manner that the drive shaft is equidistantly offset from an inner perimeter of the chamber; providing a drum concentrically attached directly to the drive shaft and journaled thereabout while remaining spaced inwardly from the inner perimeter of the chamber; providing a plurality of non-linear arms with opposed proximal and distal ends directly engaged with the drum and partially seated within the passageway respectively; providing a plurality of paddles anchored to distal ends of the arms and contiguously brushing along a major portion of an outer perimeter of the passageway; providing a crank handle statically mated with the drum and configured in such a manner that an outer edge of the crank handle extends beyond said outer perimeter of said chamber; uniformly articulating the drum along clockwise and counter clockwise directions based upon a user input; synchronously rotating the paddles and the arms; and effectively discharging a residual quantity of the body fluid from the sack and reducing the likelihood of undesirable odor and contamination over extended periods of time by expelling the air and the body fluid along the clockwise and counter clockwise directions.

The method further includes the steps of: maintaining each of the paddles and the arms spaced from the inlet and outlet ports while the paddles are abutting against the outer perimeter; and evenly discharging the air and the body fluid along the chamber.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

It is noted the purpose of the foregoing abstract is to enable the U.S. Patent and Trademark Office and the public generally, especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

### BRIEF DESCRIPTION OF THE SEVERAL VIEWS OF THE DRAWING

The novel features believed to be characteristic of this invention are set forth with particularity in the appended claims. The invention itself, however, both as to its organization and method of operation, together with further objects and advantages thereof, may best be understood by reference to the following description taken in connection with the accompanying drawings in which:

FIG. 1 is a front perspective view of a urine storing apron, in accordance with the present invention;

FIG. 2 is a rear perspective view of the urine storing apron, in accordance with the present invention;

FIG. 3 is a rear perspective view of the urine storing apron, showing the folding motion of the apron portion, in accordance with the present invention;

FIG. 4 is a side elevational view of the urine storing apron, in accordance with the present invention;

FIG. 5 is an enlarged view of the simultaneous discharge mechanism, as seen in FIG. 4; and

FIG. 6 is an enlarged, side and cross sectional view of the simultaneous discharge mechanism, in accordance with the present invention.

### DETAILED DESCRIPTION OF THE INVENTION

The present invention will now be described more fully hereinafter with reference to the accompanying drawings, in which a preferred embodiment of the invention is shown. This invention may, however, be embodied in many different forms and should not be construed as limited to the embodiment set forth herein. Rather, this embodiment is provided so that this application will be thorough and complete, and will fully convey the true scope of the invention to those skilled in the art. Like numbers refer to like elements throughout the figures.

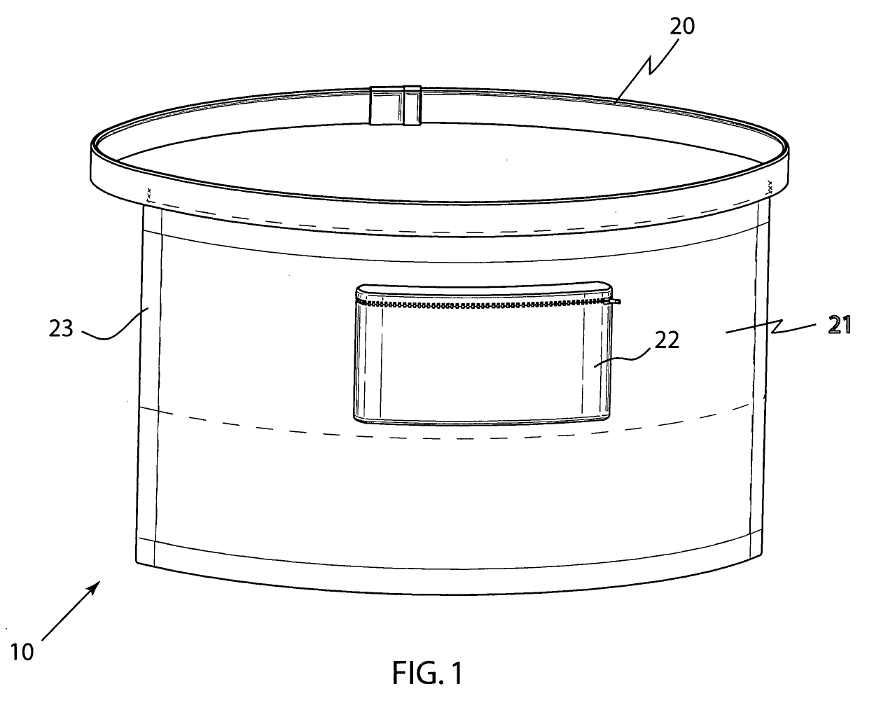

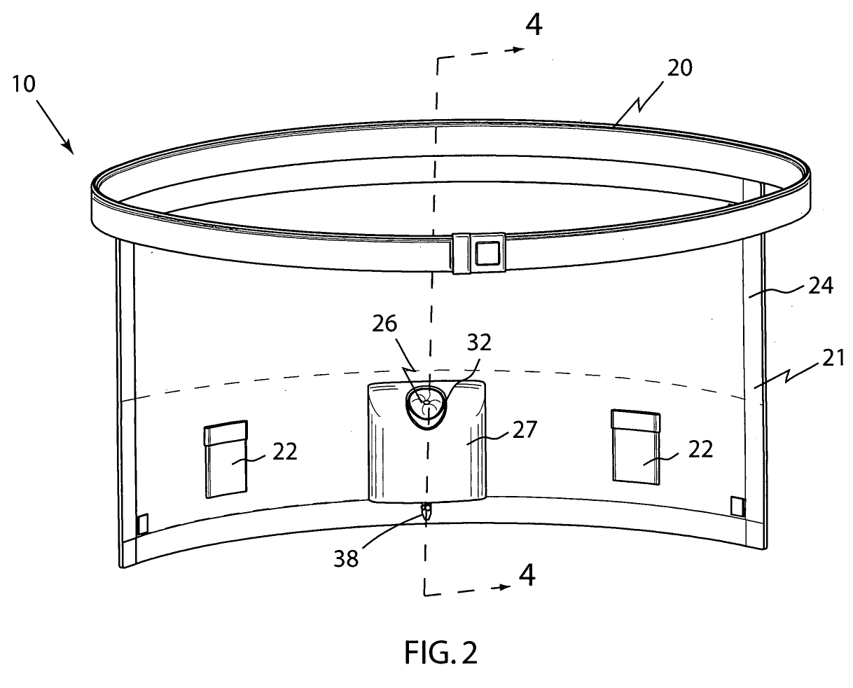

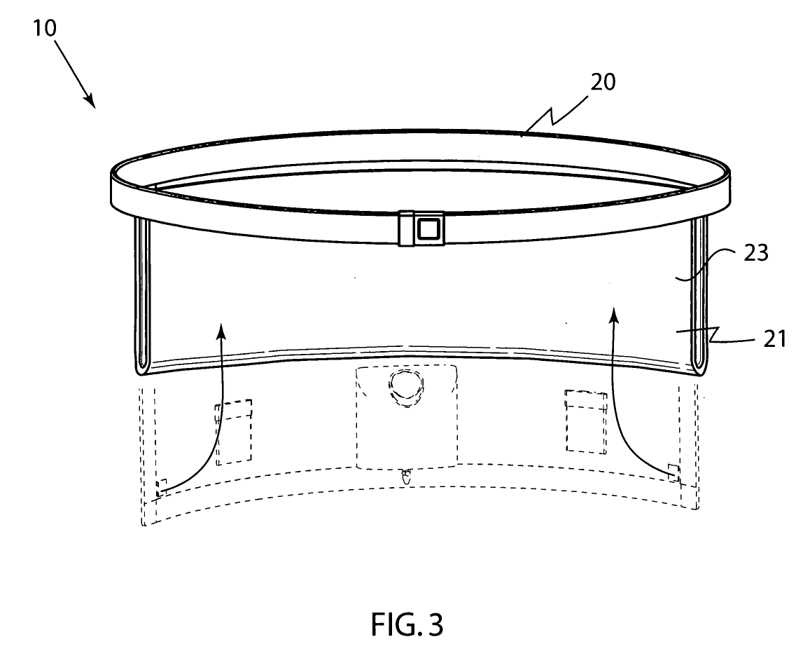

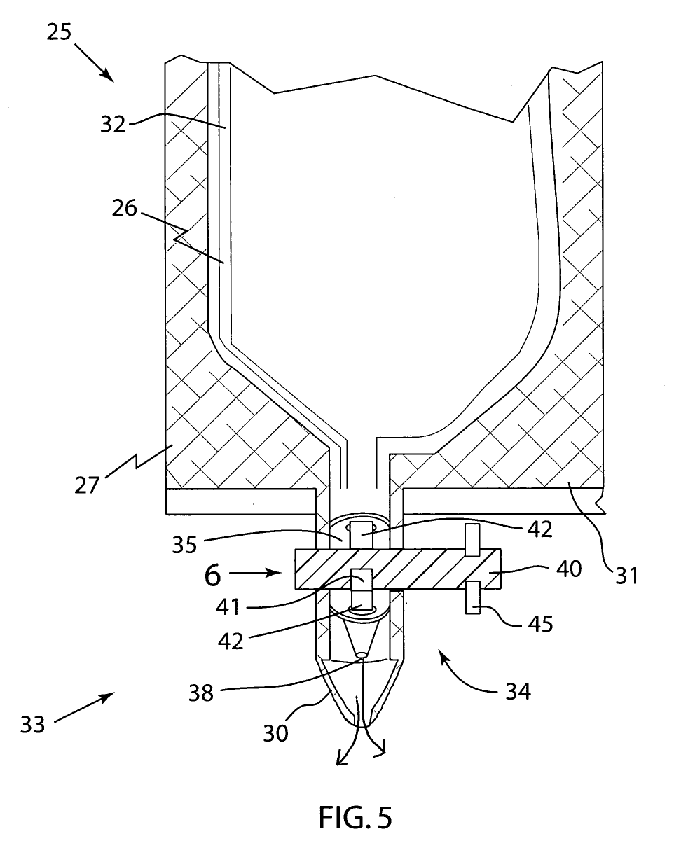

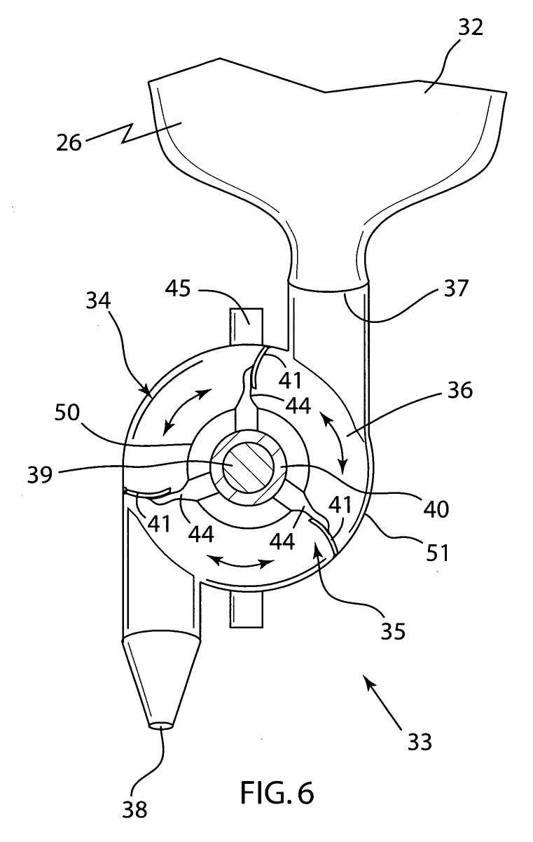

The apparatus of this invention is referred to generally in FIGS. 1-6 by the reference numeral 10 and is intended to protect a urine storing apron. It should be understood that the apparatus 10 may be used to store many different types of excretions and should not be limited to use with storing only those types of excretions mentioned herein.

Referring initially to FIGS. 1, 2, 3 and 4, a urine storing apron 10 includes a flexible belt 20, a flexible apron 21 coupled to the belt 20 and extending downwardly therefrom, and a plurality of pouches 22 attached to anterior and posterior sides 23, 24 of the apron 21.

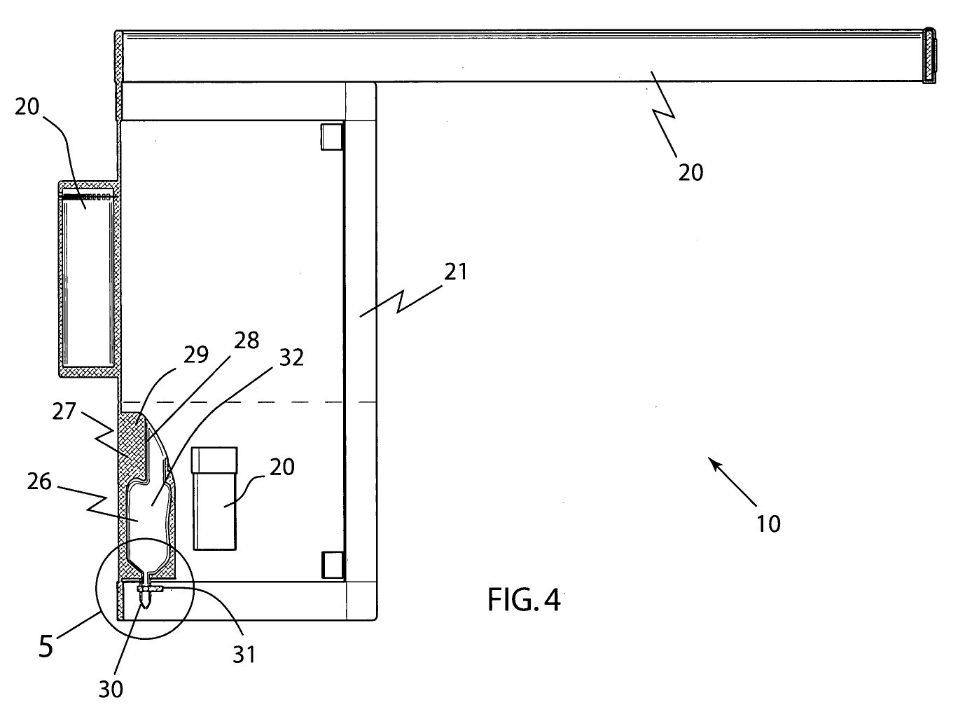

Referring to FIGS. 4, 5 and 6, the apparatus 10 further includes a mechanism 25 for storing the body fluid inside one of the pouches 27. Such a body fluid storing mechanism 25 includes a flexible sack 26 seated within the one pouch 27 and adaptable for receiving and storing the body fluid therein. Such a flexible sack 26 has an open top end 28 contiguously oriented with a top opening 29 of the one pouch 27 for receiving the body fluid therethrough, and further has an open bottom end 30 disposed subjacent to a bottom-most end 31 of the one pouch 27 for directing the body fluid away from the one pouch 27. An upper portion 32 of the sack 26 houses the body fluid and is hidden inside of the one pouch 27 which is essential such that the body fluid is not visible from an exterior of the one pouch 27 while the open bottom end 30 of the sack 26 is visible from exterior of the one pouch 27.

Referring again to FIGS. 4, 5 and 6, the apparatus 10 further includes a mechanism 33 for simultaneously discharging air and the body fluid from the one pouch 27 by rotationally articulating a portion of the simultaneous discharge mechanism 33 about an axis registered orthogonal to an exiting travel path of the body fluid. Such a simultaneous discharging mechanism 33 includes a bidirectional valve 34 with a hollow chamber 35 intermediately configured between the open top and bottom ends 28, 30 of the sack 26. Such a chamber 35 has a circular passageway 36 formed therein, and the chamber 35 further has oppositely seated inlet and outlet ports 37, 38 wherein the inlet port 37 is in fluid communication with the upper portion 32 of the sack 26 and the outlet portion 38 is in fluid communication with the open bottom end 30 of the sack 26 respectively.

The simultaneous discharging mechanism 33 further includes a drive shaft 39 medially housed at a center of the bidirectional valve 34 and disposed exterior of the passageway 36 in such a manner that the drive shaft 39 is equidistantly offset from an inner perimeter 50 of the chamber 35. A drum 40 is concentrically attached directly, without the use of intervening elements, to the drive shaft 39 and journaled thereabout while remaining spaced inwardly from the inner perimeter 50 of the chamber 35, and a plurality of non-linear arms 41 has opposed proximal and distal ends directly engaged, without the use of intervening elements, with the drum 40 and partially seated within the passageway 36 respectively. A plurality of paddles 44 is anchored to distal ends 43 of the arms 41 and contiguously brushes along a major portion of an outer perimeter 51 of the passageway 36. Also, a crank handle 45 is statically mated with the drum 40 and configured in such a manner that an outer edge of the crank handle 45 extends beyond said outer perimeter of said chamber to thereby uniformly articulate the drum 40 along clockwise and counter clockwise directions based upon a user input. The outlet port of the chamber is importantly located upstream of the open bottom end of the sack for effectively expelling the body fluid downwardly towards a converging path prior to exiting the sack and thereby ensuring that the body fluid is effectively discharged. Such an arrangement between the outlet port and the open bottom end of the sack is critical for ensuring the body fluid is discharged along a concentrated path and not a diverging path that will make it difficult to discharge the body fluid in space confined areas such as a toilet opening or a urinal, for example.

The paddles 44 and the arms 41 rotate in sync and thereby expel the air and the body fluid along the clockwise and counter clockwise directions so that the user can effectively discharge a residual quantity of the body fluid from the sack 26 and reduce the likelihood of undesirable odor and contamination over extended periods of time. Further, each of the paddles 44 are statically coupled to corresponding ones of the arms 41 and are provided with curvilinear front and rear sides for effectively forcing the air and the body fluid from the inlet port to the outlet port 37, 38 while traveling along the passageway. The inlet and outlet ports 37, 38 are diametrically offset from a center of the chamber 35 which is important such that the air and the body fluid travel approximately half-way along the passageway 36 after entering the inlet port 37 and exiting through the outlet port 38. Each of the paddles 44 and the arms 41 are coextensively shaped for evenly discharging the air and the body fluid along the chamber while remaining spaced from the inlet and outlet ports 37, 38 respectively.

The apparatus is preferably an apron just for men that is designed to serve as a urination receptacle. The apron may measure 26″ in length and width, as an example. The apron may be outfitted with 2 snap-buckle straps, reinforced with strips of hook and loop fastener, for example, to facilitate security to the person. The apron is preferably equipped with a handy plastic pouch, which serves to house urine should the need arise. This is accomplished via an integrally attached, condom-like apparatus leading into the pouch. In addition, a handy drain valve is positioned at the bottom of the pouch to facilitate emptying. A separate pouch may flank each side of this center unit and serves as storage space for additional accessories. On the front panel of the apron, a convenient zippered compartment may be offered as storage space for personal items, such as a wallet, keys, cell phone, and the like.

The present invention, as claimed, provides the unexpected and unpredictable benefit of an apparatus that is convenient and easy to use, is lightweight yet durable in design, and provides males who suffer from bladder control problems with a comfortable, private means of relieving themselves when a restroom is not readily available. Such an apparatus provides males with incontinence a simple and effective means of relieving themselves as well as protection from accidents. An apron with accessories that are preferably configured to fit the male genitalia, this practical apparatus offers males a comfortable and secure fit, eliminating the need to rely on products that are conditioned more for women. In this manner, men who suffer from incontinence and similar conditions are able to go about their daily routines with confidence, ensured that the apparatus allows them to avoid the embarrassment of walking around in visibly stained clothing. While the apparatus is primarily for household consumers, it will also be a helpful tool for medical patient care. The present invention is inexpensive, simple to use, and comfortable for the user.

In use, a method for assisting a user to collect and safely discharge body fluids on-demand includes the steps of: providing a flexible belt 20; providing a flexible apron 21 coupled to the belt 20 and extending downwardly therefrom; providing a plurality of pouches 22 attached to anterior and posterior sides 23, 24 of the apron 21; storing the body fluid inside one of the pouches 27; and simultaneously discharging air and the body fluid from the one pouch 27 by providing and rotationally articulating a bidirectional valve 34 about an axis registered orthogonal to an exiting travel path of the body fluid.

In use, the method further includes the steps of: providing a flexible sack 26 seated within the one pouch 27 and adaptable for receiving and storing the body fluid therein; and depositing the body fluid through an open top end 28 contiguously oriented with a top opening of the one pouch 27.

In use, the method further includes the steps of: providing a hollow chamber 35 intermediately configured between the open top and bottom ends 28, 30 of the sack 26 and having a circular passageway 36 formed therein; and providing inlet and outlet ports 37, 38 in fluid communication with the upper portion 32 of the sack 26 and the bottom end 30 of the sack 26 respectively.

In use, the method further includes the steps of: providing a drive shaft 39 medially housed at a center of the bidirectional valve by disposing the drive shaft 39 exterior of the passageway 36 in such a manner that the drive shaft 39 is equidistantly offset from an inner perimeter of the chamber 35; providing a drum 40 concentrically attached directly, without the use of intervening elements, to the drive shaft 39 and journaled thereabout while remaining spaced inwardly from the inner perimeter of the chamber 35; providing a plurality of non-linear arms 41 with opposed proximal and distal ends directly engaged with the drum 40 and partially seated within the passageway 36 respectively; providing a plurality of paddles 44 anchored to distal ends of the arms 41 and contiguously brushing along a major portion of an outer perimeter of the passageway 36; providing a crank handle 45 statically mated with the drum 40 and configured in such a manner that an outer edge of the crank handle 45 extends beyond said outer perimeter of said chamber 35; uniformly articulating the drum 40 along clockwise and counter clockwise directions based upon a user input; synchronously rotating the paddles 44 and the arms 41; and effectively discharging a residual quantity of the body fluid from the sack 26 and reducing the likelihood of undesirable odor and contamination over extended periods of time by expelling the air and the body fluid along the clockwise and counter clockwise directions.

In use, the method further includes the steps of: maintaining each of the paddles 44 and the arms 41 spaced from the inlet and outlet ports 37, 38 while the paddles 44 are abutting against the outer perimeter; and evenly discharging the air and the body fluid along the chamber 35.

While the invention has been described with respect to a certain specific embodiment, it will be appreciated that many modifications and changes may be made by those skilled in the art without departing from the spirit of the invention. It is intended, therefore, by the appended claims to cover all such modifications and changes as fall within the true spirit and scope of the invention.

In particular, with respect to the above description, it is to be realized that the optimum dimensional relationships for the parts of the present invention may include variations in size, materials, shape, form, function and manner of operation. The assembly and use of the present invention are deemed readily apparent and obvious to one skilled in the art.

What is claimed as new and what is desired to secure by Letters Patent of the United States is:

1. A urine storing apron for assisting a user to relieve himself from body fluids on-demand, said urine storing apron comprising:
a belt;
an apron coupled to said belt;
a plurality of pouches attached to said apron;
means for storing the body fluid inside one of said pouches; and
means for simultaneously discharging air and the body fluid from said one pouch by rotationally articulating a portion of said simultaneous discharge means about an axis registered orthogonal to an exiting travel path of the body fluid.

2. The urine storing apron of claim 1, wherein said body fluid storing means comprises:
a flexible sack seated within said one pouch and being adaptable for receiving and storing the body fluid therein, said flexible sack having an open top end contiguously oriented with a top opening of said one pouch for receiving the body fluid therethrough, said sack further having an open bottom end disposed subjacent to a bottom-most end of said one pouch for directing the body fluid away from said one pouch;
wherein an upper portion of said sack houses the body fluid and is hidden inside of said one pouch such that the body fluid is not visible from an exterior of said one pouch while said open bottom end of said sack is visible from exterior of said one pouch.

3. The urine storing apron of claim 2, wherein said simultaneous discharging means comprises:
a bidirectional valve having a hollow chamber intermediately configured between said open top and bottom ends of said sack, said chamber having a circular passageway formed therein, said chamber further having oppositely seated inlet and outlet ports wherein said inlet port is in fluid communication with said upper portion of said sack and said outlet portion is in fluid communication with said open bottom end of said sack respectively.

4. The urine storing apron of claim 3, wherein said simultaneous discharging means further comprises:
a drive shaft medially housed at a center of said bidirectional valve and disposed exterior of said passageway in such a manner that said drive shaft is equidistantly offset from an inner perimeter of said chamber;
a drum concentrically attached directly to said drive shaft and journaled thereabout while remaining spaced inwardly from said inner perimeter of said chamber;
a plurality of non-linear arms having opposed proximal and distal ends directly engaged with said drum and seated within said passageway respectively;
a plurality of paddles anchored to distal ends of said arms and contiguously brushing along a major portion of an outer perimeter of said passageway; and
a crank handle mated with said drum and configured in such a manner that an outer edge of said crank handle extends beyond said outer perimeter of said chamber to thereby uniformly articulate said drum along clockwise and counter clockwise directions based upon a user input;
wherein said paddles and said arms rotate in sync and thereby expel the air and the body fluid along the clockwise and counter clockwise directions so that the user can effectively discharge a residual quantity of the body fluid from said sack and reduce the likelihood of undesirable odor and contamination over extended periods of time.

5. The urine storing apron of claim 4, wherein each of said paddles are statically coupled to corresponding ones of said arms and are provided with curvilinear front and rear sides for effectively forcing the air and the body fluid from said inlet port to said outlet port while traveling along said passageway.

6. The urine storing apron of claim 3, wherein said inlet and outlet ports are diametrically offset from a center of said chamber such that the air and the body fluid travel approximately half-way along said passageway after entering said inlet port and exiting through said outlet port.

7. The urine storing apron of claim 4, wherein each of said paddles and said arms are coextensively shaped for evenly discharging the air and the body fluid along said chamber while remaining spaced from said inlet and outlet ports respectively.

8. A urine storing apron for assisting a user to relieve himself from body fluids on-demand, said urine storing apron comprising:
a flexible belt;
a flexible apron coupled to said belt and extending downwardly therefrom;
a plurality of pouches attached to anterior and posterior sides of said apron;
means for storing the body fluid inside one of said pouches; and
means for simultaneously discharging air and the body fluid from said one pouch by rotationally articulating a portion of said simultaneous discharge means about an axis registered orthogonal to an exiting travel path of the body fluid.

9. The urine storing apron of claim 8, wherein said body fluid storing means comprises:
a flexible sack seated within said one pouch and being adaptable for receiving and storing the body fluid therein, said flexible sack having an open top end contiguously oriented with a top opening of said one pouch for receiving the body fluid therethrough, said sack further having an open bottom end disposed subjacent to a bottom-most end of said one pouch for directing the body fluid away from said one pouch;
wherein an upper portion of said sack houses the body fluid and is hidden inside of said one pouch such that the body fluid is not visible from an exterior of said one pouch while said open bottom end of said sack is visible from exterior of said one pouch.

10. The urine storing apron of claim 9, wherein said simultaneous discharging means comprises:
a bidirectional valve having a hollow chamber intermediately configured between said open top and bottom ends of said sack, said chamber having a circular passageway formed therein, said chamber further having oppositely seated inlet and outlet ports wherein said inlet port is in fluid communication with said upper portion of said sack and said outlet portion is in fluid communication with said open bottom end of said sack respectively.

11. The urine storing apron of claim 10, wherein said simultaneous discharging means further comprises:
a drive shaft medially housed at a center of said bidirectional valve and disposed exterior of said passageway in such a manner that said drive shaft is equidistantly offset from an inner perimeter of said chamber;
a drum concentrically attached directly to said drive shaft and journaled thereabout while remaining spaced inwardly from said inner perimeter of said chamber;
a plurality of non-linear arms having opposed proximal and distal ends directly engaged with said drum and partially seated within said passageway respectively;
a plurality of paddles anchored to distal ends of said arms and contiguously brushing along a major portion of an outer perimeter of said passageway; and
a crank handle statically mated with said drum and configured in such a manner that an outer edge of said crank handle extends beyond said outer perimeter of said chamber to thereby uniformly articulate said drum along clockwise and counter clockwise directions based upon a user input;
wherein said paddles and said arms rotate in sync and thereby expel the air and the body fluid along the clockwise and counter clockwise directions so that the user can effectively discharge a residual quantity of the body fluid from said sack and reduce the likelihood of undesirable odor and contamination over extended periods of time.

12. The urine storing apron of claim 11, wherein each of said paddles are statically coupled to corresponding ones of said arms and are provided with curvilinear front and rear sides for effectively forcing the air and the body fluid from said inlet port to said outlet port while traveling along said passageway.

13. The urine storing apron of claim 10, wherein said inlet and outlet ports are diametrically offset from a center of said chamber such that the air and the body fluid travel approximately half-way along said passageway after entering said inlet port and exiting through said outlet port, said outlet port of said chamber being located upstream of said open bottom end of said sack for expelling the body fluid downwardly towards a converging path prior to exiting said sack and thereby ensuring that the body fluid is effectively discharged.

14. The urine storing apron of claim 11, wherein each of said paddles and said arms are coextensively shaped for evenly discharging the air and the body fluid along said chamber while remaining spaced from said inlet and outlet ports respectively.

15. A method for assisting a user to collect and safely discharge body fluids on-demand, said method comprising the steps of:
a. providing a flexible belt;
b. providing a flexible apron coupled to said belt and extending downwardly therefrom;
c. providing a plurality of pouches attached to anterior and posterior sides of said apron;
d. storing the body fluid inside one of said pouches; and
e. simultaneously discharging air and the body fluid from said one pouch by providing and rotationally articulating a bidirectional valve about an axis registered orthogonal to an exiting travel path of the body fluid.

16. The method of claim 15, wherein step d. comprises the steps of:
providing a flexible sack seated within said one pouch and being adaptable for receiving and storing the body fluid therein; and
depositing the body fluid through an open top end contiguously oriented with a top opening of said one pouch.

17. The method of claim 16, wherein step e. comprises the steps of:
providing a hollow chamber intermediately configured between said open top and bottom ends of said sack and having a circular passageway formed therein; and
providing inlet and outlet ports in fluid communication with said upper portion of said sack and said a bottom end of said sack respectively.

18. The method of claim 17, wherein step e. comprises the steps of:
providing a drive shaft medially housed at a center of said bidirectional valve by disposing said drive shaft exterior of said passageway in such a manner that said drive shaft is equidistantly offset from an inner perimeter of said chamber;
providing a drum concentrically attached directly to said drive shaft and journaled thereabout while remaining spaced inwardly from said inner perimeter of said chamber;
providing a plurality of non-linear arms having opposed proximal and distal ends directly engaged with said drum and seated within said passageway respectively;
providing a plurality of paddles anchored to distal ends of said arms and contiguously brushing along a major portion of an outer perimeter of said passageway;
providing a crank handle mated with said drum and configured in such a manner that an outer edge of said crank handle extends beyond said outer perimeter of said chamber;
uniformly articulating said drum along clockwise and counter clockwise directions based upon a user input;
synchronously rotating said paddles and said arms; and
effectively discharging a residual quantity of the body fluid from said sack and reducing the likelihood of undesirable odor and contamination over extended periods of time by expelling the air and the body fluid along the clockwise and counter clockwise directions.

19. The method of claim 18, further comprising the steps of:
maintaining each of said paddles and said arms spaced from said inlet and outlet ports while said paddles are abutting against the outer perimeter; and
evenly discharging the air and the body fluid along said chamber.
